(19) United States
(12) Patent Application Publication
(10) Pub. No.: US 2018/0092816 A1
(43) Pub. Date: Apr. 5, 2018

(54) Topical Hydrogen-Containing Skin Care Products

(71) Applicant: N.V. Perricone LLC, San Francisco, CA (US)

(72) Inventors: Nicholas V. Perricone, Meriden, CT (US); Christopher C. Caires, Berkeley, CA (US)

(21) Appl. No.: 15/720,681

(22) Filed: Sep. 29, 2017

Related U.S. Application Data
(60) Provisional application No. 62/402,031, filed on Sep. 30, 2016.

(51) Int. Cl. A61K 8/19; A61K 8/55; A61K 8/02; A61K 8/14; A61Q 19/08

(52) U.S. Cl. CPC A61K 8/19; A61K 8/553; A61K 8/0295; A61K 2800/56; A61Q 19/08; A61K 2800/22; A61K 8/14

(57) ABSTRACT

A method for improving the appearance of the skin, moisturizing skin and/or providing an antioxidant effect to skin by administering to skin a composition comprising an effective amount of a hydrogen-generating agent or molecular hydrogen and a carrier having a phosphatidylcholine component entrapping the hydrogen-generating agent or molecular hydrogen. A skin care product has an effective amount of molecular hydrogen and/or a hydrogen-generating agent; and a carrier entrapping them. Desirably, the composition is an aqueous base, and the hydrogen-generating agent and the carrier form a first phase of the composition, and an emulsifier forms a second phase of the composition.

### FIELD OF THE INVENTION

The present invention relates to cosmetic preparations containing hydrogen that are effective for the treatment and prevention of conditions of aging skin, as an anti-oxidant, and a moisturizer. The invention further comprises novel methods of preparing and storing hydrogen containing cosmetic compositions suitable for the treatment and prevention of conditions of aging skin, as an anti-oxidant, and a moisturizer.

### BACKGROUND OF THE INVENTION

Human skin is constantly directly exposed to the air, solar radiation, environmental pollutants, or other mechanical and chemical damage agents, which can induce the generation of free radicals, particularly oxygen free radicals including reactive oxygen species (ROS) generated by a person's cellular systems. Cellular damage in the skin can develop due to many different factors, including: ionizing radiation, severe physical and psychological stress, alcohol intake, poor nutrition, overeating, environmental pollution, and exposure to UV radiation (UVR). It is estimated that among all these environmental factors, UVR contributes up to 80% of cellular damage. UV-induced generation of ROS in the skin develops oxidative stress, when the ROS rate of formation exceeds the antioxidant defense ability of the target cell. The primary mechanism by which UVR initiates molecular responses in human skin is via photochemical generation of ROS, mainly formation of superoxide anion (O(2)(-)(.), hydrogen peroxide (H(2)O(2)), hydroxyl radical (OH(.), and singlet oxygen ((1)O(2)). Over time, the presence of ROS can damage skin cells, causing conditions of aging skin.

Hydrogen and hydrogen generating compounds such as metal hydrides are known to react with oxygen free radicals. Hydrogen gas is only slightly soluble in water. Nevertheless, submersion in a bath filled with Hydrogen gas (H2)-dissolved water or electrolyzed reduced water has been reported to repress UV damage by ROS-scavenging and promotion of type-I collagen synthesis in dermis. E.g., S. Kato et al., Journal of Photochemistry and Photobiology B: Biology 106 (2012) 24-33.

Hydrogen can be generated or extracted by many methods. A typical process for hydrogen generation is electrolysis of water, which generates hydrogen molecules and oxygen. Hydrogen may also be generated by reacting metal hydrides with water. Known substances that react with water to generate a hydrogen gas include magnesium metal, alkaline earth metal hydrides such as magnesium hydride, and metal borohydride salts such as sodium borohydride.

U.S. Pat. No. 4,439,416 discusses the use of metal hydrides, such as sodium and potassium borohydride, to produce exothermically self-heated shaving cream compositions. The hydride is employed in a liquid medium and packaged separately from a reducible medium such that the two agents can be combined and activated at the time of use.

US 2008/0292541 discloses a hydrogen generating composition comprised of a hydrogen compound, such as alkali metal hydrides, alkali earth metal hydrides and metal borohydride salts, embedded in a water-soluble solid compound such as a polyethylene glycol and organic acid. The hydrogen generating agent dissolves slowly in water to generate hydrogen gas.

US 2009/0074735 discloses metal hydrides, hydrogen gas, or any other material that readily reacts with free radicals for incorporation into compositions meant to interact with and neutralize free radicals. However, no appreciable details as to which particular compounds or ingredients, at what ranges they should be used, or with what ingredients they should be formulated is disclosed or discussed.

US 2010/0272789 discloses a hydrogen generator for treatment or prevention of cosmetic and dermatological conditions comprising a metal hydride as a first agent, a protic solvent as a second agent, and a container for mixing the first and second agents so as to generate active hydrogen or hydrogen molecules.

US 2013/0323190 discloses magnesium hydride (MgH₂) having a particle diameter of not more than 2 μm for formulation into topical compositions used to whiten, lighten spots, and treat wounds and burns on skin. The compositions comprise 1-5% wt/vol magnesium hydride suspended in oil, such as horse oil for application to skin. It is discussed that the particles may also be incorporated into cream or gel form; however, no specific ingredients other than oils are disclosed.

US 2014/0247689 discloses methods and apparatus that can be used to increase hydrogen concentration in water, in various beverages, and in other hydrogen absorbing materials through a sealed hydrogen gas releasing chamber. The chamber connecting wall is made of materials that have good hydrogen permeability and that can withhold gas pressure differences across the wall. In particular, the materials used for the chamber connecting wall can be rubber, silicone rubber, vinyl methyl silicone rubber, and phenyl vinyl methyl silicone rubber. The gas producing chamber can tolerate an acidic reaction environment, high gas pressure, and hot temperature of beverages. The hydrogen gas is generated inside the hydrogen gas producing chamber by a hydrogen gas producing system. The hydrogen gas producing system includes a cartridge to dispense hydrogen gas producing chemicals. Example chemicals used inside the cartridge for hydrogen gas generation are magnesium and citric acid.

Magnesium hydride reacts with water or acidic water to generate hydrogen. However, the reaction is not suitable for practical use because the reaction rate is so low. The use of magnesium hydride or sodium borohydride as a hydrogen generating agent often requires acidic water for increasing the reaction rate. Hydrogen compounds with increased hydrogen generation rates are likely to be fine powders and are likely to be hygroscopic, and therefore are more difficult to use or process because of their greater potential for oxidation of the hydrogen, leading to potential risks of fire or explosion.

For example, calcium hydride and lithium hydride rapidly react with water, and immediately generate hydrogen upon contact with water. While useful in a product that is mixed and then immediately used, they are not suitable for formulation into cosmetic compositions that require stability and a predictable shelf life. a. In the same way, sodium borohydride rapidly reacts with acidic water, so that the rate of hydrogen generation is substantial during an initial period but declines in subsequent periods.

A problem to be solved by the present invention is to provide stable, practical hydrogen containing or hydrogen generating compositions that can be used as topical cosmetic compositions to treat and prevent conditions of skin aging, and/or as an antioxidant, and/or for skin moisturization. Another problem to be solved by the present invention is to provide hydrogen containing or hydrogen generating topical cosmetic compositions that are easy to handle and apply as cosmetic treatments

### SUMMARY OF THE INVENTION

It is an object of the invention to provide new cosmetic product formulations containing hydrogen.

It is an object of the invention to provide new methods of formulating cosmetic products containing hydrogen.

It is an object of the invention to provide a method and apparatus for formulating stabilized cosmetic products containing hydrogen where the hydrogen is contained in a shelf stable base.

It is an object of the invention to provide a method and apparatus for formulating and delivering cosmetic products containing hydrogen where the hydrogen is generated immediately prior to use of the product.

It is an object of the invention to provide new and improved methods of providing the skin with antioxidant protection.

It is an object of the invention to provide new and improved methods of providing the skin with moisturization.

It is an object of the invention to provide new and improved methods of treating and preventing the appearance of wrinkles and fine lines, dryness, dullness or lack of radiance of skin, and for treating and/or preventing the appearance of exaggerated lines and wrinkles, sagging, discoloration, or redness and blotchiness of aging skin.

The foregoing problems and objectives are solved by the present invention, which provides hydrogen containing and hydrogen generating cosmetic compositions that comprise hydrogen or a hydrogen source and a cosmetically stable carrier.

In a first embodiment, gaseous hydrogen is bubbled into a first phase. The first phase is saturated with hydrogen at a rate that is significantly higher than the 1.6 ppm saturation limit of hydrogen in water. The first phase can then be added to a formulated skincare product during the final step to impart a final concentration of hydrogen between 1-100 ppm.

The first phase may comprise a lecithin, such as phosphatidylcholine. In certain embodiments, the lecithin is present in liposomes, micelles, or other vesicles containing hydrogen. The composition can take the form of a gel, a cream, a lotion, an ointment, a solution, a solid “stick,” etc., that can be rubbed or sprayed onto the skin.

In certain embodiments, the hydrogen gas or a hydride may be contained within a first phase comprising a lecithin such as phosphatidylcholine, which is combined with an aqueous base and an emulsifier. In some of those embodiments, the first phase forms discrete vesicles. For example, the first phase can form liposomes. In some embodiments, the first phase forms multilamellar liposomes. In some embodiments, the first phase forms a liquid crystal.

In some embodiments, at least some of the hydrogen is present within the first phase as a gas. In some embodiments, at least some of the hydrogen is present within the first phase bound to lecithin and/or phosphatidylcholine.

In some embodiments, a composition can be loaded with hydrogen by passing hydrogen gas through the composition under conditions wherein the composition is in a fluid state. In some embodiments, hydrogen gas is bubbled through a composition that is in a fluid state. In some embodiments, hydrogen gas is bubbled for about 5 hours or less, for example about 4-5 hours, about 3-4 hours, about 2-3 hours, about 1-2 hours, about 30-60 minutes, about 15-30 minutes, or about 5-15 minutes.

According to another set of embodiments, the composition comprises an emulsion comprising a first phase comprising hydrogen gas and lecithin, and an aqueous base and an emulsifier.

Another embodiment of the invention involves a solid source of hydrogen, desirably a hydride, added to a formulated skincare product having an aqueous base during a final step, to generate hydrogen gas “in situ” which stays suspended or dissolved in the formulated product. In preferred embodiments, the hydride is sodium borohydride.

In another embodiment, hydrogen is provided in a topical composition for treatment of aging skin where solid hydrogen material is mixed with an aqueous base when a consumer actuates a dispenser to dispense the product. Again the preferred solid hydrogen material is sodium borohydride but may be other hydrogen generating materials discussed herein.

In some embodiments, the lecithin comprises a phosphatidylcholine. In some embodiments, at least some of the phosphatidylcholine comprises a phosphatidylcholine comprising a stearic diglyceride linked to a choline ester of a phosphoric acid. In some embodiments, at least some of the phosphatidylcholine comprises a phosphatidylcholine comprising a palmitic diglyceride linked to a choline ester of a phosphoric acid. In some embodiments, at least some of the phosphatidylcholine comprises a phosphatidylcholine comprising an oleic diglyceride linked to a choline ester of a phosphoric acid. In some embodiments, at least some of the phosphatidylcholine comprises a polyenylphosphatidylcholine. In some embodiments, the polyenylphosphatidylcholine comprises linoleic acid. In some embodiments, the polyenylphosphatidylcholine comprises dilinoleoylphosphatidylcholine. In some embodiments, at least about 30 wt % of the phosphatidylcholine is a polyenylphosphatidylcholine.

In some embodiments, the first phase further comprises a transdermal penetration enhancer. In some embodiments, the transdermal penetration enhancer is present at least about 5% by weight of the composition. In some embodiments, the first phase comprises a fatty acid ester. In some embodiments, the first phase comprises ascorbyl palmitate. In some embodiments, the first phase comprises isopropyl palmitate. In some embodiments, the first phase further comprises 1,3-dimethyl-2-imidazolidinone.

In some embodiments, the composition further comprises 1,2-propanediol. In some embodiments, the composition further comprises a polyglycol. In some embodiments, the polyglycol comprises polyethylene glycol.

In some embodiments, the composition is a gel. In some embodiments, the composition is a cream. In some embodiments, the composition is substantially transparent.

In some embodiments, the composition has a viscosity of at least about 20,000 cP. In some embodiments, the composition has a viscosity of at least about 50,000 cP.

In some embodiments, the lecithin is present at least about 0.5% by weight. In some embodiments, the lecithin is present at least about 1% by weight. In some embodiments, the lecithin is present at least about 30% by weight. In some embodiments, the lecithin is present at least about 60% by weight.

In another set of embodiments, the composition is generally directed to a gel or a cream comprising hydrogen. In some embodiments, a hydrogen containing composition prepared as described herein comprises no more than 250 ppm of water. In some instances, the gel comprises no more than about 250 ppm of water. In some embodiments, the first phase of the composition comprises no more than about 250 ppm of water. In some embodiments, one or more of the materials used to prepare the composition each comprise no more than about 250 ppm of water.

In some embodiments, a composition comprising hydrogen is unexpectedly stable and can be stored long-term. Thus, for example, a composition of the invention may exhibit a long shelf life, with little loss or reaction of hydrogen.

In yet another aspect, the present invention encompasses methods of making one or more of the embodiments described herein.

Another aspect of the present invention is directed to a method of formulating a topical hydrogen composition. For example, in some embodiments, the method comprises acts of mixing a polyglycol and a phosphatidylcholine to form a phosphatidylcholine solution, and delivering gaseous hydrogen into the phosphatidylcholine solution.

The invention further comprises methods of use of the described compositions to obtain an antioxidant effect on the skin; and to obtain moisturization of the skin. Another aspect of the present invention is directed to a method for treating or improving the appearance of aging skin, including surface spots, brown spots, red areas, wrinkles and texture and other artifacts of aging skin, as well as conditions of skin dryness, dullness, loss of elasticity, lack of radiance, exaggerated lines and wrinkles, spider vessels or red blotchiness by applying to skin a composition comprising an effective amount of hydrogen and a carrier having a phosphatidylcholine component entrapping the hydrogen.

### DETAILED DESCRIPTION OF THE INVENTION

A stabilized cosmetic product is a shelf stable composition provided with molecular hydrogen in a first embodiment and/or a hydrogen-generating agent such as a hydride suspended in an aqueous base in a second embodiment. Hydrogen gas is entrapped or contained within the product as discussed herein, for example, in liquid crystal multilamellar phosphatidylcholine or liposomes. The composition may be stable and can be stored for periods of time with little or no loss or reaction of the hydrogen contained therein.

In a third embodiment, a cosmetic product is adapted for generation of hydrogen immediately prior to use of the product. The cosmetic product is an aqueous base provided with a container and system which permits the introduction of a hydrogen-generating agent such as a hydride immediately prior to use of the product.

The skin care product provided herein should be stable in the presence of hydrogen and hydrogen sources as well as compatible with these materials. The product should be stable and non-reactive with other ingredients in the system. One or more hydrogen generating agents is therefore applied in admixture with a dermatologically acceptable carrier or vehicle (e.g., as a lotion, cream, ointment, soap, stick, or the like) so as to facilitate topical application and provide additional desirable effects such as improving the appearance of skin, moisturizing of the affected skin areas, and providing antioxidant effects to the skin.

Preferred embodiments of a hydrogen-generating agent are metal hydrides, particularly alkali metal borohydrides such as sodium and potassium borohydrides. Sodium and potassium borohydrides contribute to making satisfactory cosmetic products for application to skin since their by-products are well tolerated by skin. Magnesium hydrides, including magnesium borohydride, are another preferred hydrogen generating agent. Silicon hydrides or silyl hydrides, including silanes, silenes, silynes, may also be used. A preferred form of silicon based hydrogen generating agent is polymethylhydrosiloxane.

Other less preferred hydrides include alkaline earth metal borohydrides typified by calcium borohydride; ammonium borohydride; quaternary ammonium borohydrides such as cetyl trimethylammonium borohydride; alkali metal aluminum hydrides such as sodium aluminum hydride; and earth metal aluminum hydrides such as calcium aluminum hydride. The disadvantages of certain of these compounds are discussed above. Most preferred hydride sources (H—) are R₃B where R=at least one

H.

The hydride is preferably a dry, solid material in granular or powder form but may also be in a liquid carrier in order to facilitate the commingling of the hydride with a cosmetic matrix.

The aqueous base containing the hydride in the second and third embodiments must be carefully formulated since hydrides will release hydrogen in the presence of water through the gradual decomposition of the compound. The hydride may be readily dissolved by the liquid medium or it may be suspended in a non-solvent base. In either case the hydride is sufficiently stable in the selected medium so that the hydride does not substantially decompose prior to contact with the skin. Preferably, the hydride is stably suspended in a non-solvent liquid carrier. The non-solvents that provide a stable suspension of hydride particles therein are any of those which do not dissolve the hydride and have the ability to provide a dispersion of undissolved solid particles in a liquid medium, the dispersion being of such nature that the solid particles do not settle and cake so that each small portion of the suspension will have practically the same composition. However, the non-solvents should be compatible with cosmetic preparations and those looked upon as suitable components for cosmetic compositions are preferred. Suitable non-solvents are hydrocarbon materials such as liquefied, paraffinic hydrocarbons, e.g. mineral or paraffin oil, liquid petrolatum or white mineral oil and similar mixtures of liquid hydrocarbons obtained from petroleum.

Examples of aqueous bases include water, serums, skin lotions, beauty essences, milky lotions, creams and face pack gels containing various moisture retention components or other components, for example, transdermal penetration enhancers, adjuvants, surfactants, lubricants, etc.

The aqueous base is preferably incorporated in a formulation appropriate for topical application, and particularly one which will form a film or layer on the skin to which it is applied so as to localize the application and provide some resistance to washing off by immersion in water or by perspiration and/or aid in the percutaneous delivery of the active agent(s). Many preparations are known in the art, and include lotions containing oils and/or alcohols and emollients vegetable oils, hydrocarbon oils and waxes, silicone oils, animal or marine fats or oils, glyceride derivatives, fatty acids or fatty acid esters, or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. These ingredients can be formulated into a cream, lotion, or gel, or a solid stick, by utilization of different proportions of the ingredients and/or by inclusion of thickening agents such as gums or other forms of hydrophilic colloids. One possible embodiment is a solution used to saturate a pad used to wipe affected areas; another is a cleanser; and others are lotions, creams, and gels, which are referred to herein as dermally or dermatologically acceptable carriers, and are formulated using conventional techniques known to those of ordinary skill in the art.

Any suitable amount of hydrogen may be present within a composition prepared as described herein. For example, at least about 0.3 wt %, at least about 0.5 wt %, at least about 0.7 wt %, at least about 1 wt %, at least about 1.5 wt %, at least about 2 wt %, at least about 2.5 wt %, at least about 3 wt %, at least about 5 wt % at least about 10 wt %, at least about 20 wt %, at least about 30 wt %, at least about 40 wt %, at least about 50 wt %, at least about 60 wt %, at least about 70 wt %, at least about 80 wt %, at least about 90 wt %, at least about 100 wt %, at least about 110 wt %, or at least about 120 wt % of the composition can be hydrogen, where the basis of the weight percentage is the weight of the composition before hydrogen is added. In other examples, the suitable amount of hydrogen may be at least about 0.5 ppm; at least about 1 ppm. In certain preferred embodiments, the hydrogen concentration in the final composition is 0.5 to 200 ppm; more preferably, 1 to 100 ppm; most preferably, 50-100 ppm.

In one embodiment of the invention hydrogen gas is contained within a first phase. The first phase includes a lecithin, preferably phosphatidylcholine. Thus, the compositions of the invention comprise, in certain aspects, a phase comprising phosphatidylcholine in which hydrogen is contained within or “trapped.” The phosphatidylcholine or lecithin may be processes to form vesicles, e.g., micelles or liposomes. The phosphatidylcholine or lecithin composition can be unilamellar or multilamellar in some embodiments. In some processes the phosphatidylcholine or lecithin forms a multilamellar liquid crystal.

Molecular hydrogen may be introduced into the first phase as small bubbles where it is entrapped and/or bound to lecithins or phosphatidylcholines. For example, the hydrogen may be bound to double bonds present in the lecithins or phosphatidylcholines. Phosphatidylcholine is believed to stabilize and/or contain the hydrogen. In some cases, stability of the composition can be achieved at room temperature (about 25° C.), and/or at other temperatures such as those described herein. Without wishing to be bound by any theory, it is believed that the phosphatidylcholine adopts a liquid crystal structure under such conditions, which can thereby contain the hydrogen, e.g., as small gaseous bubbles, and/or through binding with lecithins or phosphatidylcholines.

In accordance with certain aspects of the invention, the composition may be prepared by mixing a first phase and a second phase together, then passing hydrogen gas through the mixture. The first phase may comprise a lecithin such as phosphatidylcholine and/or polyenylphosphatidylcholine, e.g., PPC-enriched phosphatidylcholine, for instance, as described herein. The second phase can comprise an emulsifier, or any other components discussed herein. In some embodiments, other components are also mixed into the composition, before or after (or while) adding hydrogen, for example, transdermal penetration enhancers, adjuvants, polyglycols (e.g., PEG and/or PPG), surfactants, lubricants, etc. as discussed herein. In some embodiments, however, hydrogen may be passed through the first phase prior to mixing of the first phase with the second phase.

Hydrogen may be bubbled into the mixture under pressures such as between about 3,000 Pa and about 15,000 Pa, between about 5,000 Pa and about 10,000 Pa, or between about 6,000 Pa and about 8,000 Pa, and/or temperatures such as between about 0° C. and about 50° C., between about 20° C. and about 35° C., or about 25° C. and about 30° C. However, higher or lower pressures also may be used in some embodiments as aspects of the invention are not limited in this respect.

In certain embodiments, the hydrogen is bubbled through the mixture until the mixture begins to at least partially solidify. As an example, the viscosity of the mixture may increase to at least about 1,000 cP, at least about 2,000 cP, at least about 3,000 cP, at least about 5,000 cP, at least about 7,000 cP, at least about 10,000 cP, at least about 12,000 cP, at least about 15,000 cP, at least about 20,000 cP, at least about 30,000 cP, at least about 40,000 cP, at least about 50,000 cP, at least about 60,000 cP, at least about 70,000 cP, or at least about 80,000 cP. The hydrogen can be passed through the mixture as pure hydrogen, and/or with other gases (e.g., a noble gas, for example, argon). In some cases, a hydrogen donor may be passed into the mixture, and therein, at least some of the hydrogen donor can be converted into hydrogen. In other embodiments, however, the final composition may have lower viscosities, for example, such that the composition is liquid, or could be sprayed onto the skin.

In one set of embodiments, the hydrogen can be bubbled through the mixture to cause the viscosity of the mixture to increase. For example, the viscosity can increase until the mixture begins to form a gel, a cream, a lotion, an ointment, a solid “stick,” or the like. A cream may be, for example, a semi-solid emulsion, e.g., comprising a first phase and a second phase. The first phase may be discontinuous (e.g., comprising small droplets or vesicles, such as is discussed herein) and the second phase may be continuous, or vice versa. In some cases, however, both the first phase and the second phase are co-continuous within the mixture.

In certain embodiments, gaseous hydrogen is bubbled into a matrix. The matrix is saturated at 10,000 ppm of hydrogen which is significantly higher than the 1.6 ppm saturation limit of hydrogen in water. The matrix can then be added to a formulated skincare product during a final step to impart a final concentration of hydrogen between 1-100 ppm.

In some embodiments of the invention, a composition may be prepared as discussed above, then diluted, e.g., with a diluent, to produce a final composition. For example, a “stock” composition may be initially prepared, e.g., having a relatively high hydrogen concentration, then the stock composition diluted to produce a final composition, e.g., before use, before storage, before packaging, etc. In some embodiments, the diluent used may be a component as discussed herein (for example, forming at least a portion of the second phase), and the same or different materials than may be present in the initial composition may be used. The dilution ratio (amount of diluent added, relative to the initial composition) may be at least about 2, at least about 3, at least about 5, at least about 10, at least about 15, at least about 20, at least about 25, at least about 30, at least about 50, or at least about 100, or any other suitable factor.

As another example, the mixture may be mixed with or otherwise include adjunct ingredients, if applicable, and hydrogen may be introduced to the mixture, e.g., bubbles of hydrogen gas may be blown into the mixture until the mixture hardens to obtain the desired final composition.

As a specific non-limiting example, a hydrogen composition may be formed by preparing a matrix which is a non-liposome multilamellar liquid crystal phosphatidylcholine phase, for example, by providing a polyglycol, then introducing phosphatidyl choline into the glycol at room temperature to form a phosphatidylcholine solution. The phosphatidylcholine often comes as a solid (e.g., as a “brick” of material), and the phosphatidylcholine may be broken down into smaller pieces to aid in mixing, e.g., by “shaving” or grinding the phosphatidylcholine solid. The phosphatidylcholine solution is mixed until the phosphatidylcholine solution is substantially clear, then one may warm the phosphatidylcholine solution to 40° C., mill the warmed solution (i.e., low agitation after the initial mixing), combine siloxylated polyether and polydimethylsiloxane to form a fluid, add the fluid to the warmed solution and milling until the solution is clear, adding methyl paraben or other suitable lubricant to the solution and milling until the methyl paraben dissolves in the solution, warm water to 40° C. and adding the warmed water slowly to the solution, and then ceasing milling of the solution and “sweeping” the solution (e.g., with a sweep mixer) to cool to room temperature. Hydrogen gas can then be bubbled or otherwise introduced into the solution while cooling the solution until the solution begins to harden or becomes stiff, e.g., having the consistency of a gel or a cream, such as previously described. In some cases, the resulting composition is sealed in a container, for example, as discussed herein. Any suitable container may be used, e.g., a tube or a bottle. In addition, the composition (e.g., within the container) may be stored at room temperature, or any other suitable temperature. For example, a composition of the invention may be stored at or below 80° C., e.g., at or below room temperature (about 25° C.) or in a refrigerator (e.g., at 4° C.) for extended period of storage, for instance, to prevent hydrogen leakage or denaturing.

Although larger molecules and drugs have been stabilized in phosphatidylcholine or lecithin compositions or vesicles, it is unexpected that a small molecule such as H2 could be stabilized in such a matrix, especially when it would have been expected that a molecule as small as H2 would readily diffuse away from such compositions and/or would have reacted with oxygen that is typically present within such compositions.

In other embodiments, the invention is directed at formulations which use a hydrogen generating agent. Such embodiments use one or more hydrogen generating agents, typically as a fine grain particulate added to a formulated skincare product during a final step to generate hydrogen “in situ” after the product container is sealed so that hydrogen is available in the formulated product when it is opened. In preferred embodiments, the hydrogen generating agent is a hydride. Preferred hydrides are metal hydrides, including sodium borohydride, magnesium hydride, and silicon hydride. Polymethylhydrosiloxane is another acceptable hydrogen generating agent. Hydrogen generating agents include alkali earth metals like Mg or other hydride sources (H—) like R₃B where R=at least one H.

The hydrogen generating agents are typically provided as particulates dispersed in the composition. The hydrides are preferably fine particles. The particle size and quantity of the finely divided hydride particles will determine the concentration and time period of availability of an effective amount of hydrogen in the composition and the availability of hydrogen over time. Generally, the particle size of the hydrides will range from about 10 microns to about 100 microns although coarser or finer materials may be used. A particle size of less than about 45 microns is preferable.

Phosphatidylcholine (herein abbreviated “PC”) is a basic component of cell membrane bilayers and the main phospholipid circulating in the plasma of blood. Phosphatidylcholine typically has a phospholipid structure with a choline head group and a glycerophosphoric acid tail group. The tail group can be saturated or unsaturated. More than one tail group may be present in the phosphatidylcholine in some cases, and the tail groups may be the same or different. Specific non-limiting examples of phosphatidylcholines that could be used include one or a mixture of stearic, palmitic, margaric, and/or oleic acid diglycerides linked to a choline ester head group.

Phosphatidylcholines are a member of a class of compounds called lecithins. Typically, a lecithin is a composed of phosphoric acid, choline, fatty acids, glycerol, glycolipids, triglycerides, and/or phospholipids. In some cases, other lecithins may be used, in addition to or instead of a phosphatidylcholine. Non-limiting examples of other lecithins include phosphatidylethanolamine, phosphatidylinositol, or phosphatidic acid. Many commercial lecithin products are available, such as, for example, Lecithol®, Vitellin®, Kelecin®, and Granulestin®. Lecithin is widely used in the food industry. In some embodiments, certain compositions of the invention can contain synthetic or natural lecithin, or mixtures thereof. Natural preparations are used in some cases because they exhibit desirable physical characteristics, and/or may be economical or nontoxic. However, in other embodiments, non-natural preparations are used, or the composition can include both natural and non-natural preparations.

Generally, first and second embodiments discussed above, the molecular hydrogen and/or hydrogen generating agent is contained within a first phase comprising a lecithin, such as phosphatidylcholine, which is provided in an aqueous base, and dispersed and suspended therein by an emulsifier.

Any suitable amount of phosphatidylcholine or lecithin may be present within the composition. For example, at least about 0.25 wt %, at least about 0.5 wt %, at least about 1 wt %, at least about 2 wt %, at least about 3 wt %, at least about 5 wt %, at least about 8 wt %, at least about 10 wt %, at least about 20 wt %, at least about 30 wt %, at least about 40 wt %, at least about 50 wt %, at least about 60 wt %, at least about 70 wt %, at least about 80 wt %, or at least about 90 wt % of the entire composition can be a phosphatidylcholine or a lecithin. In some cases, the phosphatidylcholine or lecithin may be present at a concentration of no more than about 95 wt %, no more than about 90 wt %, no more than about 80 wt %, no more than about 70 wt %, no more than about 65 wt %, no more than about 60 wt %, no more than about 50 wt %, no more than about 40 wt %, no more than about 30 wt %, no more than about 20 wt %, or no more than about 10%. For instance, the phosphatidylcholine or lecithin may be present at between about 8 wt % and about 65 wt %, or between about 0 wt % and about 10 wt %, etc. One or more than one type of phosphatidylcholine or lecithin may be present.

Some delivery compositions of the present invention may contain polyenylphosphatidylcholine (herein abbreviated “PPC”). In some cases, PPC can be used to enhance epidermal penetration. The term “polyenylphosphatidylcholine,” as used herein, means any phosphatidylcholine bearing two fatty acid moieties, wherein at least one of the two fatty acids is an unsaturated fatty acid with at least two double bonds in its structure, such as linoleic acid.

Certain types of soybean lecithin and soybean fractions, for example, can contain higher levels of polyenylphosphatidylcholine, with dilinoleoylphosphatidylcholine (18:2-18:2 phosphatidylcholine) as the most abundant phosphatidylcholine species therein, than conventional food grade lecithin. Such lecithins may be useful in formulating certain delivery compositions. In some embodiments, conventional soybean lecithin may be enriched with polyenylphosphatidylcholine, for instance, by adding soybean extracts containing high levels of polyenylphosphatidylcholine. As used herein, this type of phosphatidylcholine is called “polyenylphosphatidylcholine-enriched” phosphatidylcholine (hereinafter referred to as PPC-enriched phosphatidylcholine), even where the term encompasses lecithin obtained from natural sources exhibiting polyenylphosphatidylcholine levels higher than ordinary soybean varieties. These products are commercially available, for example, from American Lecithin Company, Rhone-Poulenc and other lecithin vendors. American Lecithin Company markets its products with a “U” designation, indicating high levels of unsaturation; Rhone-Poulenc's product is a soybean extract containing about 42% dilinoleoylphosphatidylcholine and about 24% palmitoyllinoleylphosphatidylcholine (16:0 to 18:2 of PC) as the major phosphatidylcholine components. Another example of a suitable polyenylphosphatidylcholine is NAT 8729 (also commercially available from vendors such as Rhone-Poulenc and American Lecithin Company).

Any suitable amount of polyenylphosphatidylcholine may be present within the composition. For example, at least about 0.25 wt %, at least about 0.5 wt %, at least about 1 wt %, at least about 2 wt %, at least about 3 wt %, at least about 5 wt %, at least about 8 wt %, at least about 10 wt %, at least about 20 wt %, at least about 30 wt %, at least about 40 wt %, at least about 50 wt %, at least about 60 wt %, at least about 70 wt %, at least about 80 wt %, or at least about 90 wt % of the composition can be polyenylphosphatidylcholine. In some cases, the polyenylphosphatidylcholine may be present at a concentration of no more than about 95 wt %, no more than about 90 wt %, no more than about 80 wt %, no more than about 70 wt %, no more than about 65 wt %, no more than about 60 wt %, no more than about 50 wt %, no more than about 40 wt %, no more than about 30 wt %, no more than about 20 wt %, or no more than about 10%. For instance, the polyenylphosphatidylcholine may be present at between about 8 wt % and about 65 wt %. In some embodiments, at least about 20 wt %, at least about 30 wt %, at least about 40 wt %, at least about 50 wt %, at least about 60 wt %, at least about 70 wt %, at least about 80 wt %, at least about 90 wt %, or about 100 wt % of all of the phosphatidylcholine or lecithin in the composition is polyenylphosphatidylcholine.

As a specific non-limiting example of one set of embodiments, a polyenylphosphatidylcholine comprises a certain material with the trade name NAT 8729, and optionally at least one polyglycol (polyhydric alcohol of a monomeric glycol such as polyethylene glycol 200, 300, 400, 600, 1,000, 1,450, 3,350, 4,000, 6,000, 8,000 and 20,000). The composition can also comprise a PPC-enriched phosphatidylcholine material that is present within the first or second phase, e.g., comprising hydrogen or a hydrogen source. The second phase may also comprise a surfactant such as a siloxylated polyether comprising dimethyl, methyl(propylpolyethylene oxide propylene oxide, acetate) siloxane commercially available from vendors such as Dow Corning (Dow Corning 190 surfactant) and lubricant such as silicone fluids containing low viscosity polydimethylsiloxane polymers, methylparaben (p-hydroxy benzoic acid methyl ester) commercially available from vendors such as Down Corning (Dow Corning 200 silicone fluid).

While not wishing to be bound to any theory, it is believed that the phosphatidylcholine, particularly the PPC-enriched phosphatidylcholine forms a liposome or liquid crystal enveloping the molecular hydrogen or hydrogen-generating agents to create the delivery composition. The PPC-enriched phosphatidylcholine is believed to contribute to the stability of the hydrogen, for example, by shielding the hydrogen from water, and/or by enhancing its penetration into the skin.

Adjunct ingredients may be present in an amount ranging from 0.01% to about 20% by weight of the composition. For example, compositions of the invention may include adjuvants such as salts, buffering agents, diluents, excipients, chelating agents, fillers, drying agents, antioxidants, antimicrobials, preservatives, binding agents, bulking agents, silicas, solubilizers, or stabilizers. Exemplary adjunct ingredients for improvement of various conditions of skin aging include, but are not limited to one or more of: caffeine, vitamin D3, lipoic acid; a-hydroxy acids such as glycolic acid or lactic acid; ascorbic acid and its derivatives, especially fatty acid esters of ascorbic acid; tocotrienols and tocotrienol derivatives and vitamin E compositions enriched with tocotrienols or tocotrienol derivatives; and neuropeptides. Preferred adjunct agents include caffeine, tocopherol, tocopherol acetate, acetyl tyrosine, palmitoyl tripeptide-5, thiotic acid, hexylene glycol, magnesium ascorbyl phosphate and tetrahexyldecyl ascorbate. Peptides and nitric oxide, suitable as adjunct ingredients are further described in U.S. Patent Application Publication No. 20160158279, which is incorporated in its entirely by reference herein.

The first phase also comprises, in some embodiments of the invention, a fatty acid ester. Non-limiting examples include ascorbate palmitate or isopropyl palmitate. In some cases, the fatty acid ester is used as a preservative or an antioxidant. The composition can include any suitable amount of fatty acid ester, for example, at least about 1 wt %, at least about 3 wt %, at least about 5 wt %, at least about 10 wt %, at least about 20 wt %, at least about 30 wt %, at least about 40 wt %, at least about 50 wt %, etc. In some cases, no more than about 60 wt %, no more than about 50 wt %, no more than about 40 wt %, no more than about 30 wt %, no more than about 20 wt %, no more than about 18 wt %, no more than about 15 wt %, no more than about 12 wt %, or no more than about 10 wt % of the composition is fatty acid ester. For example, the composition may be between about 0 wt % and about 10 wt % fatty acid ester. The composition may include one or more than one fatty acid ester.

In certain embodiments, a composition such as those described herein can be formulated to include a second phase. Typically, the second phase is substantially immiscible with the first phase comprising phosphatidylcholine or lecithin. Two phases that are substantially immiscible are able to form discrete phases when exposed to each other at ambient conditions (e.g., 25° C. and 1 atm) for extended periods of time (e.g., at least about a day). The phases can be separate identifiable phases (e.g., one may float above the other), or in some cases, the phases are intermingled, e.g., as in an emulsion. The stability of the discrete phases may be kinetic and/or thermodynamic in nature, in various embodiments.

In one set of embodiments, the second phase may comprise an emulsifier which causes the first phase comprising phosphatidylcholine or lecithin to form a liquid crystal, and/or vesicles such as micelles or liposomes. Typically, in a liquid crystal phase, vesicular structures such as micelles, liposomes, hexagonal phases, or lipid bilayers can be formed. In some cases, multilamellar structures may be present within the liquid crystal phase, although in other cases, only unilamellar structures may be present. For example, in certain cases, the PPC-enriched phosphatidylcholine can be loosely arranged in a multilamellar fashion, with hydrogen and optional adjunct ingredients being bonded or otherwise entrapped or contained within the lipid bilayers formed therein. In some cases, the first phase (e.g., comprising PPC-enriched phosphatidylcholine) and the second phase can form a structure such as is disclosed in U.S. Pat. No. 7,182,956 to Perricone, et al. This is believed (without wishing to be bound by any theory) to form a loosely arranged, yet stable, PPC-enriched phosphatidylcholine-hydrogen complex that may allow penetration and delivery of hydrogen and optional adjunct ingredients to the skin, e.g., to the skin.

In some embodiments, the second phase may comprise a polyglycol. The polyglycol may include a polyhydric alcohol of a monomeric glycol such as polyethylene glycol (PEG) and/or polypropylene glycol (PPG). For example, the PEG or PPG may be PEG or PPG 200, 300, 400, 600, 1,000, 1,450, 3,350, 4,000, 6,000, 8,000, and 20,000, where the number indicates the approximate average molecular weight of the PEG or PPG. As is understood by those of ordinary skill in the art, a polyglycol composition often will comprise a range of molecular weights, although the approximate average molecular weight is used to identify the type polyglycol. More than one PEG and/or PPG can also be present in certain instances.

The second phase may comprise a surfactant in some embodiments. Non-limiting examples of surfactants include a siloxylated polyether comprising dimethyl, methyl(propylpolyethylene oxide propylene oxide, acetate) siloxane commercially available from vendors such as Dow Corning (Dow Corning 190 surfactant). Other examples of materials that can be used as (or within) the second phase include, but are not limited to, 1,2-propanediol, or silicone fluids containing low viscosity polydimethylsiloxane polymers, methylparaben (p-hydroxy benzoic acid methyl ester) commercially available from vendors such as Dow Corning (Dow Corning 200 silicone fluid). Still other examples include various siloxane or silicone compounds, e.g., hexamethyldisiloxane, amodimethicone, phenyltrimethicone, etc.

Additionally, purified water may be added to the second phase in some embodiments, although in other cases, little or no water is present in the second phase. For example, the first phase, the second phase, can contain less than 10%, less than 5%, less than 2%, less than 1%, or less that 0.05% (e.g., wt %) of water relative to the weight of the respective phase or of the entire composition. In some cases, the second phase may also comprise adjunct ingredients such as those described herein.

The second phase may include any one, or more than one, of the materials described above. In addition, any suitable amount of second phase can be used in accordance with various embodiments of the invention. For example, the second phase may be present at at least about 10 wt %, at least about 20 wt %, at least about 30 wt %, at least about 40 wt %, at least about 50 wt %, at least about 60 wt %, at least about 70 wt %, at least about 80 wt %, or at least about 90 wt % of the composition. In some cases, the ratio of the first phase (e.g., comprising phosphatidylcholine or lecithin) to the second phase can be at least about 1:3, at least about 1:2, at least about 1:1, at least about 2:1, at least about 3:1, or at least about 4:1, etc.

In another set of embodiments, the composition may also include one or more transdermal penetration enhancers. Examples of transdermal penetration enhancers include, but are not limited to, 1,3-dimethyl-2-imidazolidinone or 1,2-propanediol. Other examples include cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide), organic acids (e.g., citric acid); sulfoxides (e.g., dimethylsulfoxide); terpenes (e.g., cyclohexene); ureas; sugars; carbohydrates or other agents. The transdermal penetration enhancers can be present in any suitable amount within the composition. For example, at least about 10 wt %, at least about 20 wt %, at least about 30 wt %, at least about 40 wt %, or at least about 50 wt % of the composition may comprise one or more transdermal penetration enhancers. In some cases, no more than about 60 wt %, no more than about 50 wt %, no more than about 40 wt %, no more than about 30 wt %, no more than about 20 wt %, no more than about 10 wt %, no more than about 9 wt %, or no more than about 5 wt % of the composition comprises transdermal penetration enhancers. For example, the composition may have between about 0 wt % and about 5 wt % of one or more transdermal penetration enhancers.

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition.

Preservatives include, but are not limited to, C₁-C₃ alkyl parabens and phenoxyethanol, typically present in an amount ranging from about 0.1% to about 2.0% by weight percent, based on the total composition. Preferred preservatives include phenoxyethanol and/or sodium benzoate.

Emollients, typically present in amounts ranging from about 0.01% to 10% of the total composition include, but are not limited to, fatty esters, fatty alcohols, mineral oils, polyether siloxane copolymers, docosahexanoic acid (DHA) and mixtures thereof. Preferred emollients include isohexadecane, cetyl alcohol, cetearyl alcohol, stearlyl alcohol, ethylhexyl palmitate, and avocado oil.

Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Preferred humectants are glycerin and glycol derivatives, such as hexylene glycol, ethylhexylglycerin, glyceryl stearate, peg-12 glyceryl dimyristate, and caprylyl glycol and shae butter.

Emulsifiers, typically present in amounts from about 1% to about 15% by weight of the composition, include, but are not limited to, polysorbagte 20, stearic acid, cetyl alcohol, stearyl alcohol, steareth 2, steareth 20, acrylates/C10-30 alkyl acrylate crosspolymers, silicones, dimethylethanolamine (DMAE), phosphatidylcholine (PPC), docosahexanoic acid (DHA) and mixtures thereof. Some preferred emulsifiers are polysorbate 20 and dimethylaminoethanol, also known as DMAE.

Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof.

Antioxidants, typically present in an amount ranging from about 0.01% to about 0.75% by weight of the composition, include, but are not limited to, butylated hydroxy toluene (BHT); vitamin C and/or vitamin C derivatives, such as fatty acid esters of ascorbic acid, particularly ascorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; and mixtures of any of these. Particularly preferred antioxidants are those that provide additional benefits to the skin, such as ascorbyl palmitate, alpha-lipoic acid, magnesium ascorbyl phosphate and tetrahexyldecyl ascorbate.

Buffering agents are employed in many compositions. Preferably, the amount of buffering agent is one that results in compositions having a pH ranging from about 4.0 to about 6.0, more preferably from about 4.5 to about 6.0, most preferably from about 4.5 to about 5.5. Typical buffering agents are chemically and physically stable agents commonly found in cosmetics, and can include compounds that are also adjunct ingredients such as citric acid, malic acid, and glycolic acid buffers.

Preparations can include sterile aqueous or nonaqueous solutions, suspensions and emulsions. Examples of nonaqueous solvents are polypropylene glycol, polyethylene glycol, vegetable oil such as olive oil, sesame oil, coconut oil, arachis oil, peanut oil, mineral oil, organic esters such as ethyl oleate, or fixed oils including synthetic mono or di-glycerides. Aqueous solvents include water, alcoholic/aqueous solutions, emulsions or suspensions, including saline and buffered media.

Other components may also be present within the composition, in accordance with certain embodiments of the invention. For example, the composition may include volatile organic fluids, fatty acids, volatile aromatic cyclic compounds, high molecular weight hydrocarbons, or the like.

Additional ingredients and methods as disclosed in U.S. Pat. Nos. 5,376,361; 5,409,693; 5,545,398; 5,554,647; 5,574,063; 5,643,586; 5,709,868; 5,879,690; 6,191,121; 6,296,861; 6,437,004; 6,979,459; 8,609,604; 8,609,618, 9,023,801; 9,029,317; and 9,034,926; which are hereby incorporated by reference, may also be used.

In some embodiments, various compositions of the invention are formulated to be substantially clear or substantially transparent. Transparency may be useful, for instance, for product acceptance in the marketplace, e.g., when applied to the skin of a subject. However, in other embodiments, the composition is not necessarily transparent. Certain substances can be useful in providing a substantially transparent composition, for example, fatty acid esters such as ascorbate palmitate or isopropyl palmitate. In one set of embodiments, the composition may be substantially transparent such that incident visible light (e.g., have wavelengths of between about 400 nm and about 700 nm) can be transmitted through 1 cm of the composition with a loss in intensity of no more than about 50%, about 60%, about 70%, about 80%, or about 90% relative to the incident light. In some embodiments, there may be no substantial difference in the wavelengths that are absorbed by the composition (i.e., white light passing through the composition appears white), although in other cases, there can be more absorption at various wavelengths (for example, such that white light passing through the composition may appear colored).

A composition may be prepared and/or stored at any suitable temperature and under any suitable conditions. In some embodiments, for instance, a composition can be prepared and/or stored under limited or no oxygen conditions, as oxygen can react with free hydrogen to form water. For instance, the composition may be prepared and/or stored in a sealed environment (e.g., stored in a sealed container). The sealed environment (e.g., container) can be at least substantially devoid of gas, and/or contains a gaseous mixture that excludes, or at least is depleted in, oxygen. In some embodiments, an environment depleted in oxygen may have less than about 20%, less than about 15%, less than about 10%, less than about 5%, about 1% or less, about 0.1% or less, about 0.01% or less, about 0.001% or less, oxygen (e.g., as a wt % or as molar % per volume). For example, the gaseous mixture may include a noble gas, such as argon, helium, neon, etc. In one set of embodiments, the container may comprise a multi-layered metallic and/or polymeric barrier, e.g., formed from Glaminate® (American Can Company). For instance, the container may have the shape of a tube. Thus, in certain embodiments, the container is substantially resistant to oxygen permeation, nitrogen permeation, and/or carbon dioxide permeation. In certain embodiments, the container is substantially watertight, for example, such that substantially no water is absorbed by the container, or such that no water is able to pass through the container even if the container is filled with water.

A low-viscosity cosmetic such as a skin lotion may be packed in an aerosol can together with a nitrogen gas or liquefied gas as an injection gas. In the first place, the hydrogen generating agent, e.g. sodium borohydride, of the present invention is injected in an aerosol can and a skin lotion is injected from the top of the can, and an injection gas is added under pressure. The can is sealed, and thus a hydrogen containing skin lotion is readily produced. The container may not be an aerosol can. Through the use of the hydrogen generating agent of the present invention, a hydrogen gas can be directly dissolved in a cosmetic in the container of the final product. Therefore, different from a production method involving dissolution of a hydrogen gas at an intermediate stage, the present method will not cause dissipation of a hydrogen gas, and provides a cosmetic having high hydrogen content.

In cosmetic compositions adapted to be dispensed as a pressure-propelled product, the propellant used in the pressurized container can vary widely. Any propellant material generally employed in pressurized dispensing containers is suitable in the practice of the present invention, although the liquefied gases are preferable. Among those gases are the poly-halogenated lower hydrocarbons such as chlorinated and fluorinated methanes, ethanes and higher homologues, e.g. monochlorodifluoromethane, monochlorodifluoroethane, dichlorodifluoromethane, dichlorodifluoroethane, trichloromonofluoromethane and dichlorotetrafluoroethane; and the lower hydrocarbons such as propane, butane and isobutane. Compressed gases such as nitrogen, oxygen, nitrous oxide and carbon dioxide may also find use. In some instances it may be desirable to use a combination of two or more of the liquefied normally gaseous materials as a propellant in order to achieve a suitable pressure within the container and impart the desired properties of stability, propellancy, ease of delivery, etc. to the cosmetic preparations.

The amount of propellant may be varied depending upon the properties desired. In general, it need be present in an amount sufficient to propel or to eject satisfactorily the contents of the container. Typically, at least 1% and preferably 2 to 5% propellant, by weight of the total composition in the container, should be used although higher amounts may be employed if desired, e.g. up to about 10%.

In another embodiment, hydrogen is provided in a dispensing system where a hydrogen-generating agent, e.g. hydride particulates, is mixed with an aqueous base when a consumer actuates a dispenser to dispense the product. In such applications, the hydride-containing portion and aqueous base are prepared separately and only combined when the product is desired for use. The hydride-containing media can be prepared in accordance with a variety of procedures.

One method by which a hydride may be placed in a liquid medium is by dissolving the hydride in a suitable solvent medium at room temperature with stirring. When hydrides which react with water (i.e. those dissolved hydrides which react very slowly with water to yield hydrogen) are employed in an aqueous medium, or a solvent medium containing water, the hydride solution should be stabilized toward hydrolysis. Thus, a hydrolysis inhibitor such as sodium hydroxide is added to the aqueous-hydride solutions with stirring to prevent hydrolysis.

However, in the preferred embodiments, the hydride is added in particulate form. The hydride should be in finely divided form prior to suspension in the base composition. The hydride can be finely divided by ball-milling, such as by using one-half inch porcelain balls. Other ingredients such as perfumes, colorants, foam stabilizers, suspending agents and the like may be incorporated in the base composition.

In accordance with the described embodiment, the hydride and the aqueous base are kept as separate until use. In one embodiment, a hydride-containing powder is encapsulated in a foil wrap or other frangible barrier in a product cap whereby the consumer will depress the cap, punching through the foil or barrier, and releasing the contents into an aqueous base composition, instantly generating hydrogen which will be captured in the composition.

In another embodiment, the hydride containing powder or an oil based suspension of hydride powder is packaged in one container, and an aqueous base is packaged in a separate container, and the consumer is directed to combine the components prior to use.

In another embodiment, the hydride-containing part and the carrier may be placed into a container divided into two separate compartments by a common wall extending the length of the container and having a common exit. Additionally, they are packaged in separate pressurized “aerosol” containers having a common discharge device in opposed axial disposition or disposed in a side-by-side relationship. Pressurized hydride portions and pressurized carriers may be prepared in any suitable manner. In general, the respective portion is placed in a container and pressurized with propellant. Generally in the practice of methods of the invention, the topical composition is topically applied to the skin areas, such as that of the face, at predetermined intervals often as a moisturizer, lotion, or cream, it generally being the case that gradual improvement is noted with each successive application.

The term “skin” means the keratinous surfaces of skin, hair and nails. The term “skin” when used herein is in the broad sense meaning the skin of the face, body, and neck as well as the lips.

In certain aspects of the invention, a composition such as those described herein can be administered to a subject, such as a human subject, by rubbing it on the skin, e.g., in areas located at or at least within the vicinity of a desired target area. Without wishing to be bound by any theory, it is believed that phosphatidylcholine provides or facilitates delivery of hydrogen to the skin, and/or to tissues below the skin, allowing hydrogen to be delivered to a target area. In some embodiments, the composition can be applied, by rubbing the composition topically against the skin, which allows the composition (or at least, hydrogen) to be absorbed by the skin. The composition can be applied once, or more than once. For example, the composition may be administered at predetermined intervals. In some embodiments, for instance, the composition may be applied once per day, twice per day, 3 times per day, 4 times per day, once every other day, once every three days, once every four days, etc. The amount of hydrogen necessary to bring about improvement is not fixed per se, and may depend upon factors such as the desired outcome, the type and severity the disease or condition, the form of hydrogen, the concentration of hydrogen present within the composition, etc.

Thus, another aspect of the invention provides methods of administering any composition such as is discussed herein to a subject. When administered, the compositions of the invention are applied in an effective amount as a cosmetically acceptable formulation. Any of the compositions of the present invention may be administered to the subject. When administered to a subject, effective amounts will depend on the particular desired outcome. An effective amount may be determined by those of ordinary skill in the art, for instance, employing factors such as those described herein and using no more than routine experimentation.

In certain embodiments of the invention, the administration of the composition of the invention may be designed so as to result in sequential exposures to the composition over a certain time period, for example, hours, days, weeks, months, or years. This may be accomplished, for example, by repeated administrations of a composition of the invention by one or more of the methods described herein.

The methods of use involving application of a hydrogen-containing or hydrogen-generating agent in a dermatologically acceptable carrier are expected to operate as an antioxidant and bind with oxygen free radicals including reactive oxygen species (ROS), including superoxide anion (O(2)(-)(.)), hydrogen peroxide (H(2)O(2)), hydroxyl radical (OHO), and singlet oxygen ((1)O(2)). The methods of use are expected to reduce oxidative stress upon skin cells.

A consequence of the binding of hydrogen molecules with oxygen free radicals is the formation of water molecules. If hydrogen combines with oxygen in the skin it may provide significant moisturizing effects within the skin structure. Thus the methods of use involving application of a hydrogen-containing or hydrogen-generating agent in a dermatologically acceptable carrier are expected to provide substantial moisturization effects to the skin.

Aging skin is characterized histologically by cross-linking of collagen and elastin in the dermis. This results in loss of support seen clinically as sagging and wrinkling. The present invention recognizes these processes and provides methods to minimize both prospective and existing aging conditions. In particular, the present invention provides hydrogen compositions and methods of applying such topical compositions to skin to treat various conditions of aging skin. Particularly preferred embodiments involve treatment and application of topical compositions the skin of the face neck and décolletage

The methods of use involving application of a hydrogen-containing or hydrogen-generating agent in a dermatologically acceptable carrier are expected to improve the appearance of aging skin, including surface spots, brown spots, red areas, wrinkles and texture and other artifacts of aging skin, as well as conditions of skin dryness, dullness, loss of elasticity, lack of radiance, exaggerated lines and wrinkles, spider vessels or red blotchiness. The term “surface spots” refers to brown or red spots which include freckles, acne marks or scars, hyperpigmentation and vascular lesions. The term “brown spots” refers to those caused by an excess of melanin on and within the skin, these lesions include freckles, melasma, hyperpigmentation and lentigines. The term “red areas” refers to various skin conditions such as acne, rosacea, inflammation and spider veins that have apparent red structures due to the blood vessels and hemoglobin contained in the papillary dermis. The term “wrinkles” refers to fine lines, furrows, folds and creases in the skin. Wrinkles are associated with decreased skin elasticity. The term “texture” refers to gradations in the skin's color and tone and surface peaks and valleys that are analyzed to measure smoothness. The appearance of marionette lines, smile lines, deep nasolabial fold lines, crow's feet, fine lines/wrinkles, vertical lines between the eyebrows, horizontal forehead lines, sagging thin/frail skin, skin redness and dullness may be improved using the methods of the invention.

Generally in the practice of methods of the invention, topical compositions are topically applied to the skin areas, such as that of the face, at predetermined intervals often as a serum, moisturizer, lotion, or cream, it generally being the case that gradual improvement is noted with each successive application. Although immediate effects can be observed, enhanced results are observed when the topical composition is applied twice daily, preferably in the morning and evening. No adverse side effects are anticipated to be encountered. It is an advantage of the invention that compositions of the invention do not require a pharmaceutical prescription.

Although various preferred embodiments of carrier delivery systems have been discussed above, the methods of the invention encompass application of the compositions to the skin of a subject at any suitable location using any suitable method. For example, the composition may be rubbed on, poured on, applied with an applicator (e.g., a gauze pad, a swab, a bandage, etc.), or the like. In some cases, the composition can be a liquid, a gel, a cream, a lotion, an ointment, a solid “stick,” or the like, that can be applied to the skin by hand, for example, by rubbing or spraying.

Examples of products formulated according to the invention are provided below.

### Example 1

A system in which a hydride and an aqueous base are kept as separate until use. In this embodiment, an infusion powder is contained in a product cap and released when the consumer depresses the cap, releasing the infusion into a serum base. The ingredients are as follows:

Infusion: 3-O-Ethyl Ascorbic Acid, Sodium Borohydride, Gardenia Florida Fruit Extract, Maltodextrin.

Serum Base: Water, Glycerin, Ectoin, Palmitoyl Tripeptide-5, Sodium PCA, Sodium Hyaluronate, Polygonum Cuspidatum Root Extract, Camellia Sinensis Leaf Extract, Centella Asiatica Extract, PCA, Serine, Proline, Glycine, Phenylalanine, Arginine, Alanine, Threonine, Valine, Histidine, Isoleucine, Saccharide Isomerate, Chamomilla Recutita (Matricaria) Flower Extract, Glycyrrhiza Glabra (Licorice) Root Extract, Lactobacillus/Soybean Ferment Extract, Paeonia Suffruticosa Root Extract, Rosmarinus Officinalis (Rosemary) Leaf Extract, Saccharomyces/Potato Extract Ferment Filtrate, Saccharomyces Barley Seed Ferment Filtrate, Scutellaria Baicalensis Root Extract, Panthenol, Sodium Lactate, Aspartic Acid, Acrylates/Vinyl Isodecanoate Crosspolymer, Propanediol, Butylene Glycol, Citric Acid, Dunaliella Salina Extract, Sodium Hydroxide, Disodium EDTA, Sodium Benzoate, Panthenol, Phosphatidylcholine.

In a clinical efficacy evaluation of use of the product of Example 1 applied twice a day, at morning and night, 97% of subjects showed significant improvement in the appearance of fine lines and wrinkles after 1 week, and 100% of subjects showed significant improvement in the appearance of fine lines and wrinkles after 4 weeks. 83% of subjects showed significant improvement in the appearance of skin texture after 4 weeks. 90% of subjects showed significant improvement in skin firmness after 4 weeks and 87% of subjects showed significant improvement in skin elasticity after 4 weeks. 100% of subjects showed significant improvement in skin hydration immediately after use.

### Example 2

A serum which is formulated with a hydrogen-generating agent which generates hydrogen in situ such that the serum contains molecular hydrogen.

Ingredients: Water, Methyl Gluceth-20, Ascorbyl Glucoside, Betaine, Ectoin, Polysorbate 20, Potassium Hydroxide, Hydrolyzed Sodium Hyaluronate, Panthenol, Acrylatesa/Inyl Isodecanoate Crosspolymer, 1,2-Hexanediol, Caprylyl Glycol, Butylene Glycol, Lactobacillus Ferment, Allantoin, Di Potassi Um Glycyrrhizate, Phosphatidylcholine, Trisodium Ethylenediamine Disuccinate, Phenoxyethanol, Saccharide Isomerate, Sea Whip Extract, Sodium Hyaluronate, Tocopherol, Sodium Borohydride.

### Example 3

A gel which is formulated with a hydrogen-generating agent which generates hydrogen in situ such that the gel contains molecular hydrogen.

Ingredients: Water, Cyclopentasiloxane, Glycereth-26, Glycerin, Cetyl Ethylhexanoate, Sodium Potassium Aluminum Silicate, Methyl Gluceth-20, Phosphatidycholine, Sodium Hyaluronate, Sodium Borohydride, Palmitoyl Tripeptide-5, Palmitoyl Tripeptide-38, Dunaliella Salina Extract, Avena Sativa (Oat) Kernel Extract, Ectoin, Adenosine, Sodium PCA, Sodium Lactate, Paeonia Suffruticosa Root Extract, Panthenol, Scutellaria Baicalensis Root Extract, Cyclohexasiloxane, Butylene Glycol, Cetearyl Olivate, Betaine, Dimethicone, Hydroxyacetophenone, Tromethamine, Sorbitan Olivate, Carbomer, Helianthus Annuus (Sunflower) Seed Oil, Acrylates/C10-30 Alkyl Acrylate Crosspolymer, Allantoin, Saccharomyces/Viscum Album (Mistletoe) Ferment Extract, Disodium EDTA, Glyceryl Polyacrylate, Lactobacillus/Soybean Ferment Extract, Saccharomyces/Imperata Cylindrica Root Ferment Extract, Arginine, Aspartic Acid, PCA, Glycine, Alanine, Serine, Valine, Isoleucine, Proline, Threonine, Histidine, Phenylalanine, Silica, Hydroxypropyl Cyclodextrin, Titanium Dioxide (CI 77891).

### Example 4

A facial cream which is formulated with a hydrogen-generating agent which generates hydrogen in situ such that the cream contains molecular hydrogen.

Ingredients: Water, Glycerin, Caprylic/Capric Triglyceride, Dimethicone, Dicaprylyl Carbonate, Aluminum Starch Octenylsuccinate, Porphyridium Polysaccharide, Sodium PCA, Sodium Lactate, Sodium Hyaluronate, Arachidyl Alcohol, Sodium Polyacrylate Starch, Cetearyl Alcohol, Behenyl Alcohol, Cetyl Alcohol, Caprylyl Glycol, Butyrospermum Parkii (Shea Butter), Phosphatidylcholine, PCA, Sodium Borohydride, Arginine, Dimethyl MEA, Aspartic Acid, Arachidyl Glucoside, Carbomer, Cetearyl Glucoside, Pullulan, Disodium EDTA, Glycine, Alanine, Serine, Valine, Isoleucine, Proline, Threonine, Histidine, Phenylalanine, Ethylhexylglycerin, Phenoxyethanol.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention.

1. A method comprising: administering to skin of a subject a composition comprising an effective amount of a hydrogen-generating agent or molecular hydrogen, and a phosphatidylcholine component entrapping the hydrogen-generating agent or molecular hydrogen, to improve the appearance of the skin of the subject.

2. The method of claim 1 wherein the method improves the appearance of one or more of aging skin, surface spots, brown spots, red areas, wrinkles, texture, skin dryness, dullness, loss of elasticity, lack of radiance, exaggerated lines, spider vessels and red blotchiness.

3. The method of claim 1, wherein the composition comprises an aqueous base, and a hydrogen-generating agent and the phosphatidylcholine component comprise a first phase of the composition.

4. The method of claim 3, wherein the composition is provided with an effective amount of a hydrogen-generating agent comprising a hydride.

5. The method of claim 4, wherein the hydride comprises sodium borohydride.

6. The method of claim 4, wherein the hydride is a particulate entrapped in a liposome or a multilamellar liquid crystal.

7. The method of claim 1, wherein the composition comprises an aqueous base, and molecular hydrogen and the phosphatidylcholine component comprise a first phase of the composition.

8. The method of claim 7, wherein the molecular hydrogen is entrapped in a liposome or a multilamellar liquid crystal.

9. A method of moisturizing skin, comprising:
applying to skin of a subject a composition comprising an effective amount of a hydrogen-generating agent or molecular hydrogen and a carrier having a phosphatidylcholine component entrapping the hydrogen-generating agent or molecular hydrogen, to generate moisture in the skin to improve the appearance of the skin of the subject, whereby hydrogen is provided in the skin by the hydrogen-generating agent or molecular hydrogen and the hydrogen combines with oxygen free radicals in the skin to provide moisture in the skin.

10. The method of claim 9, wherein the composition comprises an aqueous base, and the hydrogen-generating agent and the carrier comprise a first phase of the composition, and an emulsifier comprises a second phase of the composition.

11. The method of claim 10, wherein the composition is provided with an effective amount of a hydrogen-generating agent comprising a hydride.

12. The method of claim 11, wherein the hydride comprises sodium borohydride.

13. The method of claim 11, wherein the hydride is a particulate entrapped in a liposome or a multilamellar liquid crystal.

14. The method of claim 10, wherein the composition is provided with an effective amount of molecular hydrogen entrapped in a liposome or a multilamellar liquid crystal.

15. A method of providing an antioxidant effect to skin, comprising:
applying to skin of a subject a composition comprising an effective amount of a hydrogen-generating agent or molecular hydrogen entrapped in a phosphatidylcholine component whereby hydrogen is provided in the skin by the hydrogen-generating agent or molecular hydrogen and the hydrogen combines with oxygen free radicals in the skin to provide an antioxidant effect to the skin.

16. The method of claim 15, wherein the composition comprises an aqueous base, and the hydrogen-generating agent and phosphatidylcholine component comprise a first phase of the composition, and an emulsifier comprises a second phase of the composition.

17. The method of claim 16, wherein the composition is provided with an effective amount of a hydrogen-generating agent comprising a hydride.

18. The method of claim 17, wherein the hydride comprises sodium borohydride.

19. The method of claim 17, wherein the hydride is a particulate entrapped in a liposome or a multilamellar liquid crystal.

20. The method of claim 15, wherein the composition is provided with an effective amount of molecular hydrogen entrapped in a liposome or a multilamellar liquid crystal.

21. A skin care product comprising:
an effective amount of a hydrogen-generating agent;
a carrier entrapping the hydrogen-generating agent.

22. The skin care product of claim 21, wherein the hydrogen-generating agent is a hydride.

23. The skin care product of claim 22, wherein the hydride is sodium borohydride.

24. The skin care product of claim 22, wherein the carrier is a liposome or a multilamellar liquid crystal and the hydride is entrapped in the carrier.

25. The skin care product of claim 21, wherein the skin care product comprises an aqueous base, and the hydrogen-generating agent and the carrier comprise a first phase of the composition, and an emulsifier comprises a second phase of the composition.

26. The skin care product of claim 25 wherein the carrier comprises a phosphatidylcholine component.

27. A skin care product comprising:
an effective amount of molecular hydrogen, and optionally, a hydrogen-generating agent;
a carrier entrapping the molecular hydrogen.

28. The skin care product of claim 27, wherein the carrier is a liposome or a multilamellar liquid crystal.

29. The skin care product of claim 27, wherein the skin care product comprises an aqueous base, and the molecular hydrogen and the carrier comprise a first phase of the composition, and an emulsifier comprises a second phase of the composition.

30. The skin care product of claim 29 wherein the carrier comprises a phosphatidylcholine component.

31. A skin care product comprising:
an aqueous composition;
an effective amount of a hydrogen-generating agent stored adjacent to and separately from the aqueous composition whereby the hydrogen-generating agent is mixed with the aqueous composition when the skin care product is to be used on skin.

32. The skin care product of claim 31, wherein the hydrogen-generating agent is a hydride.

33. The skin care product of claim 32, wherein the hydride is sodium borohydride.

34. The skin care product of claim 31, further comprising a liposome or a multilamellar liquid crystal carrier entrapping the hydride.
